(19) United States
(12) Patent Application Publication
(10) Pub. No.: US 2007/0032791 A1
(43) Pub. Date: Feb. 8, 2007

(54) EXPANDABLE SUPPORT DEVICE AND METHOD OF USE

(76) Inventor: E. Skott GREENHALGH, Wyndmoor, PA (US)

Correspondence Address: LEVINE BAGADE HAN LLP, 2483 EAST BAYSHORE ROAD, SUITE 100, PALO ALTO, CA, 94303, US

(21) Appl. No.: 11/457,772

(22) Filed: Jul. 14, 2006

Related U.S. Application Data
(60) Provisional application No. 60/699,577, filed on Jul. 14, 2005.

(51) Int. Cl. A61F 2/30

(52) U.S. Cl. 606/61

(57) ABSTRACT

An expandable support device, such as an ultra thin expanding stent device, for tissue repair is disclosed. The expandable support device can be used to repair hard or soft tissue, such as bone or vertebral discs. A method of repairing tissue is also disclosed. The expandable support device can have a substantially flat top plate and a substantially flat bottom plate. The top plate can be attached to the bottom plate by expandable struts.

### CROSS-REFERENCE TO RELATED APPLICATIONS

This application claims the benefit of U.S. Provisional Application No. 60/699,577 filed 14 Jul. 2005, which is herein incorporated by reference in its entirety.

### BACKGROUND OF THE INVENTION

This invention relates to devices for providing support for biological tissue, for example to repair spinal compression fractures, and methods of using the same.

Vertebroplasty is an image-guided, minimally invasive, nonsurgical therapy used to strengthen a broken vertebra that has been weakened by disease, such as osteoporosis or cancer. Vertebroplasty is often used to treat compression fractures, such as those caused by osteoporosis, cancer, or stress.

Vertebroplasty is often performed on patients too elderly or frail to tolerate open spinal surgery, or with bones too weak for surgical spinal repair. Patients with vertebral damage due to a malignant tumor may sometimes benefit from vertebroplasty. The procedure can also be used in younger patients whose osteoporosis is caused by long-term steroid treatment or a metabolic disorder.

Vertebroplasty can increase the patient's functional abilities, allow a return to the previous level of activity, and prevent further vertebral collapse. Vertebroplasty attempts to also alleviate the pain caused by a compression fracture.

Vertebroplasty is often accomplished by injecting an orthopedic cement mixture through a needle into the fractured bone. The cement mixture can leak from the bone, potentially entering a dangerous location such as the spinal canal. The cement mixture, which is naturally viscous, is difficult to inject through small diameter needles, and thus many practitioners choose to “thin out” the cement mixture to improve cement injection, which ultimately exacerbates the leakage problems. The flow of the cement liquid also naturally follows the path of least resistance once it enters the bone - naturally along the cracks formed during the compression fracture. This further exacerbates the leakage.

The mixture also fills or substantially fills the cavity of the compression fracture and is limited to certain chemical composition, thereby limiting the amount of otherwise beneficial compounds that can be added to the fracture zone to improve healing. Further, a balloon must first be inserted in the compression fracture and the vertebra must be expanded before the cement is injected into the newly formed space.

A vertebroplasty device and method that eliminates or reduces the risks and complexity of the existing art is desired. A vertebroplasty device and method that is not based on injecting a liquid directly into the compression fracture zone is desired.

### BRIEF SUMMARY OF THE INVENTION

Devices for providing support for biological tissue as disclosed. The devices can be used, for example, to repair spinal compression fractures, vertebral disc decompression, spinal fusion, or combinations thereof. Methods of using the devices are disclosed.

An expandable support device for performing spinal repair is disclosed. The support device can have a top plate and a bottom plate. The top plate and/or the bottom plate can be substantially flat. The support device can have at least one strut. The strut can be expandably attach the first plate to the second plate. The strut can be foldable.

The strut can be independently foldable to vary the separation between the first plate and the second plate.

The first and second plates can define a longitudinal axis therebetween. The strut can be oriented at an intersection angle with respect to the longitudinal axis. The intersection angle can be about 90 degrees when the expandable support device is expanded into a deployed configuration.

The strut can have at least one thinning. The thinning can be more susceptible to deformation that the remainder of the strut. The thinning can be located about halfway along the length of the strut.

The top and/or bottom plates can have one or more openings therethrough.

A method for deploying an expandable support device in a spine is disclosed. The expandable support device can have a longitudinal axis, a first end and a second end. The method can include deploying the expandable support device into a target site, for example, when the expandable support device is in a compressed configuration.

The method can include expanding the expandable support device. Expanding the expandable support device can include applying forces to the spinal repair device such that the first end and the second end angularly rotate with respect to the longitudinal axis. The first end can be substantially parallel to the second end before the expanding. The first end can be substantially parallel to the second end after the expanding.

The target site can include a vertebral body, a vertebral end-plate, a vertebral disc, or combinations thereof.

Expanding the expandable support device can include increasing a height of the device while a width of the device remains unchanged. Expanding the expandable support device can include increasing a width of the device while a height of the device remains unchanged.

Deploying the expandable support device can include inserting a guide pin to or near the target site. Deploying can include advancing the expandable support device over the guide pin.

Deploying the expandable support device can include deploying the spinal repair device through a tube, such as a catheter or delivery pipe. The tube can have a lumen having a circular, oval, square, or rectangular cross-sectional shape.

### BRIEF DESCRIPTION OF THE DRAWINGS

FIG. 1 is a perspective view of a variation of the ultra thin expanding stent device in a contracted configuration

FIG. 2 is a side view of the variation of the ultra thin expanding stent device of FIG. 1.

FIG. 3 is an end view of the variation of the ultra thin expanding stent device of FIG. 1.

FIG. 4 is a perspective view of the variation of the ultra thin expanding stent device of FIG. 1 in an expanded configuration.

FIG. 5 is a perspective view of a variation of the ultra thin expanding stent device.

FIG. 6 is a side view of the variation of the ultra thin expanding stent device of FIG. 5.

FIG. 7 is a perspective view of a variation of the ultra thin expanding stent device in an expanded configuration.

FIG. 8 is a side view of the variation of the ultra thin expanding stent device of FIG. 7.

FIG. 9 is a side view of the variation of the ultra thin expanding stent device of FIG. 7 in a contracted configuration.

FIG. 10 is an end view of the variation of the ultra thin expanding stent device of FIG. 7 in a contracted configuration.

FIGS. 11 through 13 illustrate side views of various variations of the ultra thin expanding stent device.

FIGS. 14 and 15 illustrate a variation of a method for using a delivery system for the ultra thin expanding stent device.

FIGS. 16 through 18 illustrate a variation of a method for accessing a damage site in the vertebra.

FIG. 19 illustrates various variations of methods for deploying the ultra thin expanding stent device to the vertebral column.

FIGS. 20 through 25 illustrate a variation of a method for deploying the ultra thin expanding stent device into the damage site in the vertebra.

FIGS. 26 and 27 illustrate side views of various variations of a deployment tool for the ultra thin expanding stent device.

FIG. 28 illustrates a variation of a method for expanding the ultra thin expanding stent device using the deployment tool of FIGS. 26.

FIG. 29 illustrates a variation of a method for expanding the ultra thin expanding stent device using the deployment tool of FIG. 27.

FIG. 30 illustrates a variation of a method of expanding the ultra thin expanding stent device using the deployment tool of FIG. 26.

FIG. 31 illustrates a variation of a method of expanding the ultra thin expanding stent device using the deployment tool of FIGS. 27.

FIGS. 32 and 33 illustrate a variation of a method for deploying the ultra thin expanding stent device into the damage site in the vertebra.

### DETAILED DESCRIPTION

An expandable support device 110 is disclosed for tissue treatment, such as for bone compression fractures and other types of fractures. The support device can be a stent. The stent can be described herein as ultra thin, but can be of any thickness. The support device can be used to perform vertebroplasty. The support device can be used as a partial or complete vertebra replacement. The support device can be used as partial or complete vertebral disc replacement. The support device can be used for vertebra fixation.

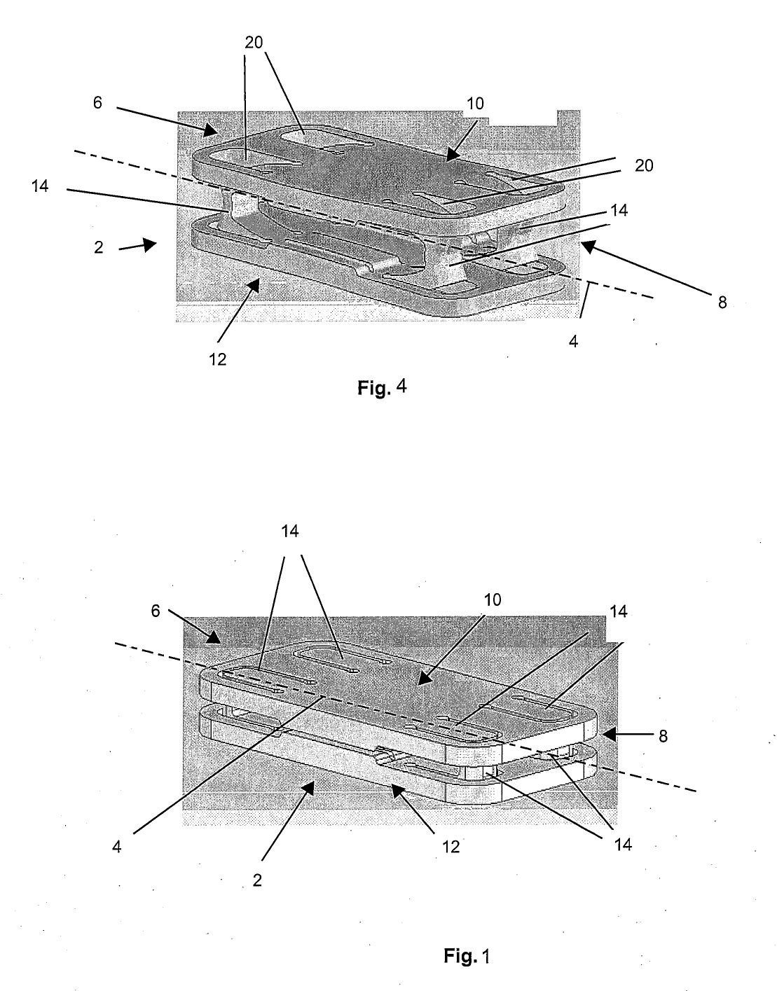

FIGS. 1 through 3 illustrate that the ultra thin expanding stent device 2 can be in a contracted configuration. The ultra thin expanding stent device 2 can be expandable in only one dimension. The ultra thin expanding stent device 2 can be biocompatible. The ultra thin expanding stent device 2 can have any configuration. The ultra thin expanding stent device 2 can be used for methods, described in the P001 Patent Application.

The ultra thin expanding stent device 2 can have a longitudinal axis 4. The ultra thin expanding stent device can have a first end and a second end 8. The ultra thin expanding stent device 2 can have plates, for example a first plate and a second plate, such as a top plate 10 and a bottom plate 12. The plates can be parallel to the longitudinal axis 4. The plates can be parallel to each other. The plates can be non-parallel to the other plates and/or to the longitudinal axis 4. The plates can be flat or curved. The curved plates can be convex and/or concave with respect to the longitudinal axis 4. The ends of the plates can be configured to dissect and/or penetrate soft and/or hard tissue, for example during implantation and deployment. The ends of the plates can be sharpened. The plates can be of uniform thickness.

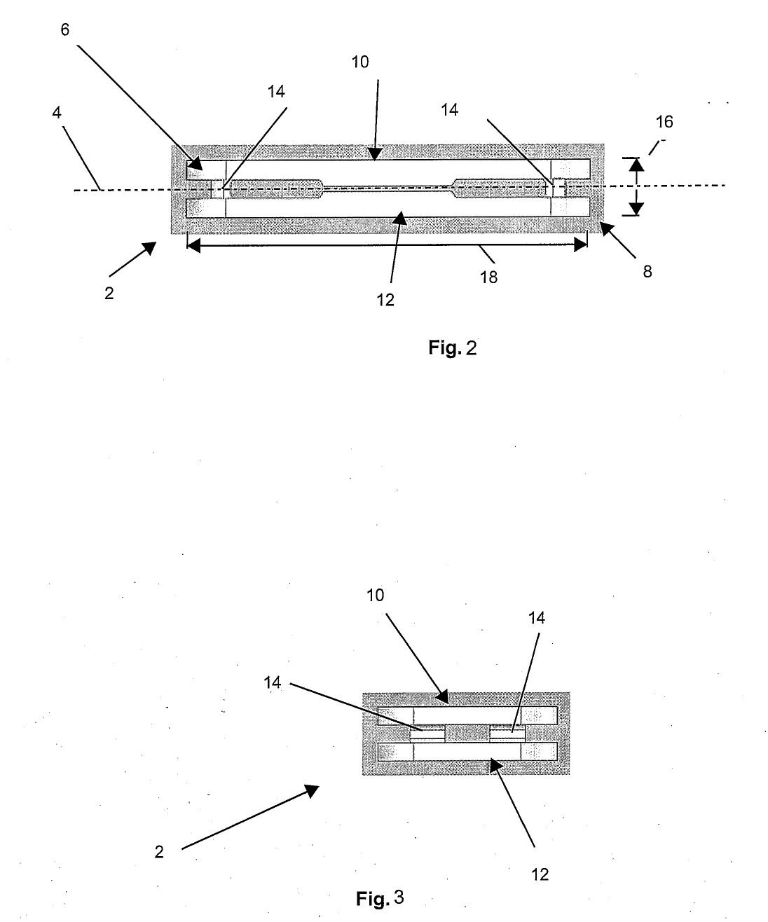

The ultra thin expanding stent device 2 can have a length, for example as illustrated in FIG. 2. The length can be from about 0.5 cm (0.2 in.) to about 2.4 cm (0.94 in.), for example about 2.2 cm (0.87 in.). The ultra thin expanding stent device 2 can have an unexpanded height 16. The unexpanded height 16 can be from about 0.2 cm (0.08 in.) to about 1 cm (0.4 in.), for example about 0.3 cm (0.1 in.).

The surfaces of the plates can be configured to increase and/or decrease friction. The surfaces can be capable of an interference fit with another object, such as a second ultra thin expanding stent device 2. The surfaces can be integral with or attached to teeth, knurled surfaces, coatings, snaps, latches, locks, slides, grooves, slots, tabs, hooks or combinations thereof.

The ultra thin expanding stent device 2 can have struts 14. The struts 14 can be attached to, or integral with, one or more plates. For example, the struts 14 can be integral with the top plate 10 and the bottom plate 12. The struts 14 can have folded sections. The folded sections can be attached to, or integral with (as shown), one or more plates. The strut 14 can have two folded sections.

The folded sections can extend from the remainder of the strut 14 toward the center of the ultra thin expanding stent device 2. The ultra thin expanding stent device 2 can have from about 2 struts 14 to about 50 struts 14, for example about four struts 14, also for example about six struts 14.

The struts 14 can have a strut length 18. The strut length 18 can be from about 10% of the ultra thin expanding stent device 2 length to about 50 percent of the ultra thin expanding stent device 2 length.

The struts 14 can be uniform in thickness along their length. The struts 14 can have a thickening, thinning and/or divot at one or more sections along the length of the strut 14. The strut 14 can have a thickening, thinning and/or divot at a folding apex. The thickening, thinning and/or divot can inhibit, facilitate and/or control folding.

The plates can have plate openings 20. For example, the top 10 and bottom plates 12 can have plate openings 20. The struts 14 can be recessed in the plate openings 20, for example, when the ultra thin expanding stent device 2 is in the unexpanded configuration.

FIG. 4 illustrates that the struts 14 can rotate or fold out of the plate openings 20, for example, when the ultra thin expanding stent device 2 is in an expanded configuration.

The ultra thin expanding stent devices 2 can have textured and/or porous surfaces for example, to increase friction against bone surfaces, and/or promote tissue ingrowth. The ultra thin expanding stent devices 2 can be coated with a bone growth factor, such as a calcium base. The plate openings 20 can be configured to increase friction against the bone surface and/or promote tissue ingrowth.

The ultra thin expanding stent device 2 can be covered by a thin metal screen. The thin metal screen can expand and/or open when the ultra thin expanding stent device 2 expands.

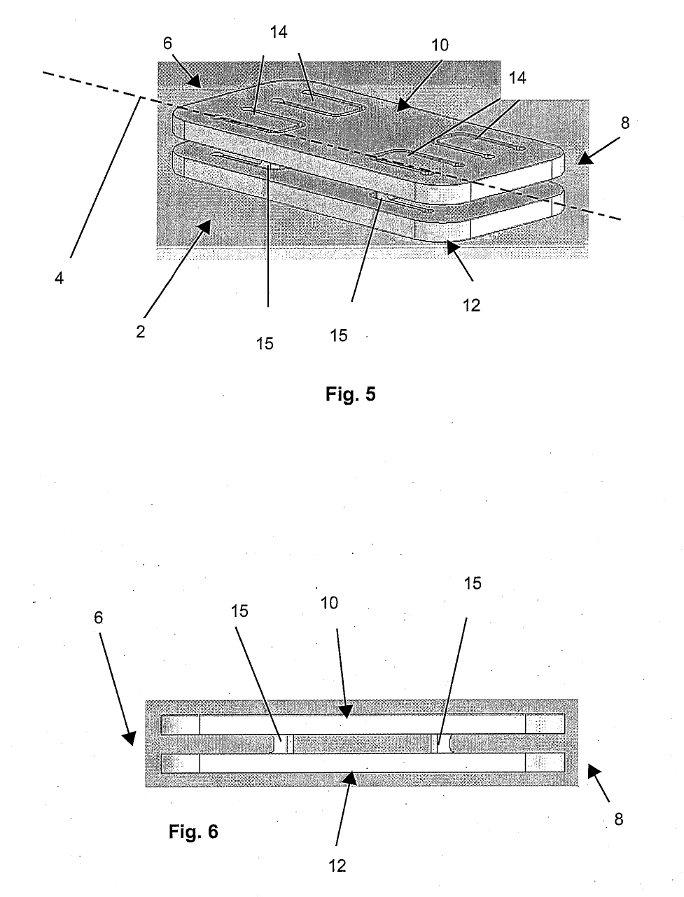

FIGS. 5 and 6 illustrate that the folded sections of the struts 14 can extend from the remainder of the strut 14 away from the center of the ultra thin expanding stent device 2.

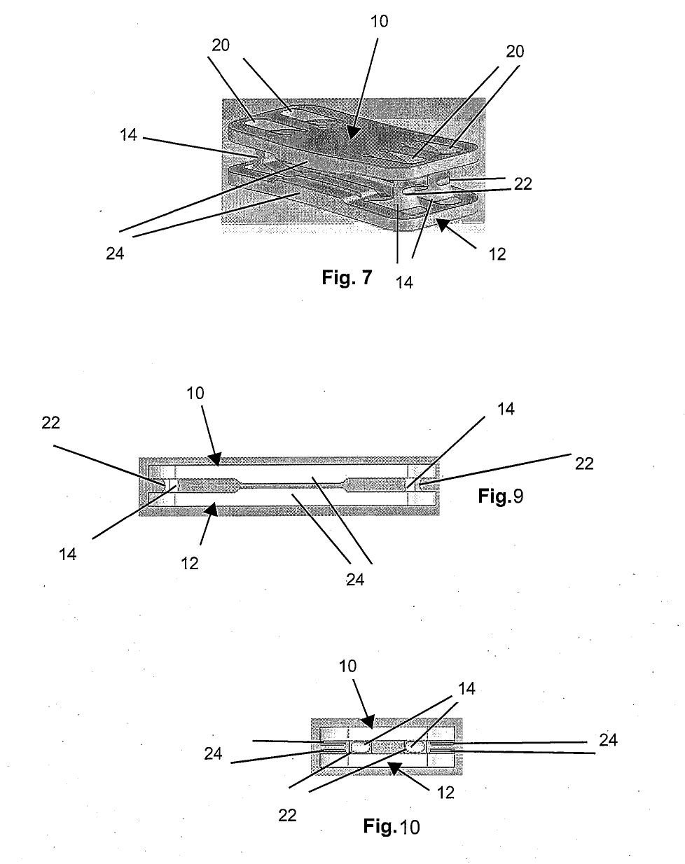

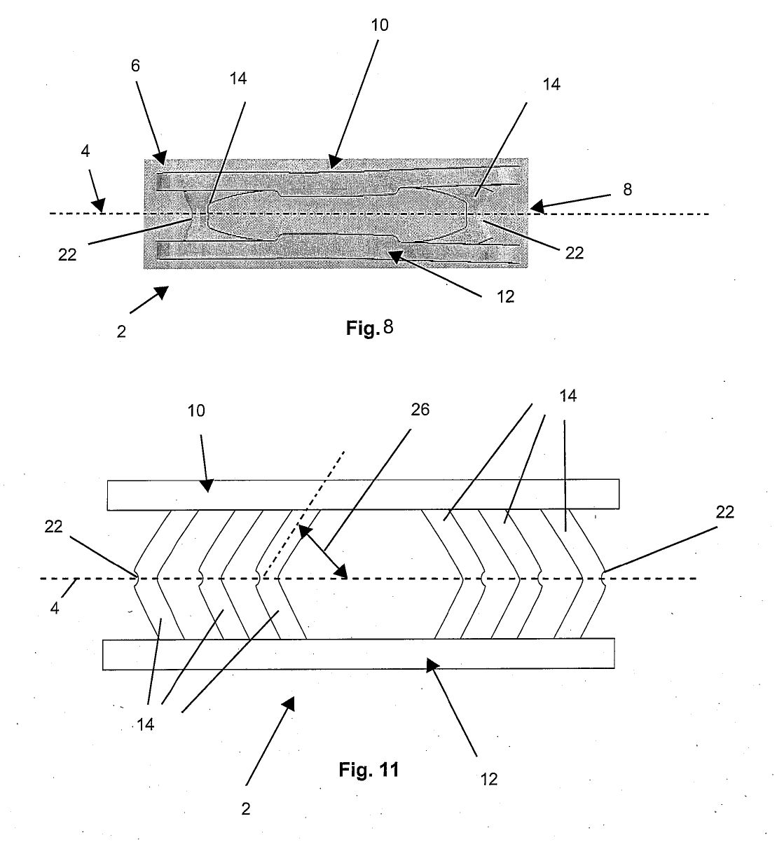

FIGS. 7 and 8 illustrate that the strut 14 can have one or more engagement thickenings, thinnings and/or divots 22. The engagement thickenings, thinnings and/or divots 22 can be configured to engage a deployment tool 30 and/or to inhibit, facilitate and/or control folding.

FIGS. 7 through 10 illustrate that the plates can have reinforcement buttresses 24. The reinforcement buttresses 24 can extend from the plates. The reinforcement buttresses 24 can be configured to be thickened portions of the plates. The reinforcement buttresses 24 can be integral with, and/or attached to, the plates.

FIGS. 11 illustrates that the ultra thin expanding stent device 2 can have expanded intersection angles 26. The expanded intersection angles 26 can be from about 45° to about 90°, for example about 70°, also for example about 90°.

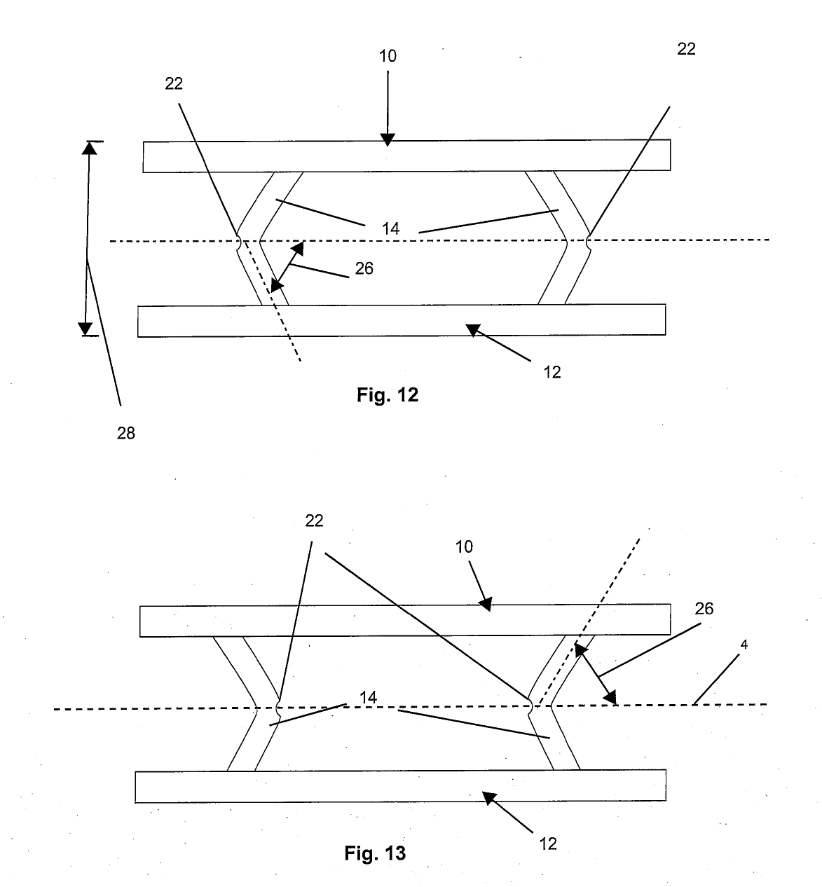

FIG. 12 illustrates that, for example when in the expanded configuration, the ultra thin expanding stent device 2 can have an expanded height 28, for example as illustrated by FIG. 12. The expanded height 28 can be from about 0.3 cm (0.1 in.) to about 2.5 cm (0.98 in.), for example about 2 cm (0.8 in.).

FIG. 13 illustrates that the expanded intersection angle 26 can always be measured as less than 90°.

Any or all elements of the ultra thin expanding stent device 2 and/or other devices or apparatuses described herein can be made from, for example, a single or multiple stainless steel alloys, nickel titanium alloys (e.g., Nitinol), cobalt-chrome alloys (e.g., ELGILOY® from Elgin Specialty Metals, Elgin, Ill.; CONICHROME® from Carpenter Metals Corp., Wyomissing, Pa.), nickel-cobalt alloys (e.g., MP35N® from Magellan Industrial Trading Company, Inc., Westport, Conn.), molybdenum alloys (e.g., molybdenum TZM alloy, for example as disclosed in International Pub. No. WO 03/082363 A2, published 9 Oct. 2003, which is herein incorporated by reference in its entirety), tungsten-rhenium alloys, for example, as disclosed in International Pub. No. WO 03/082363, polymers such as polyethylene teraphathalate (PET)/polyester (e.g., DACRON® from E. I. Du Pont de Nemours and Company, Wilmington, Del.), polypropylene, (PET), polytetrafluoroethylene (PTFE), expanded PTFE (ePTFE), polyether ether ketone (PEEK), nylon, polyether-block co-polyamide polymers (e.g., PEBAX® from ATOFINA, Paris, France), aliphatic polyether polyurethanes (e.g., TECOFLEX® from Thermedics Polymer Products, Wilmington, Mass.), polyvinyl chloride (PVC), polyurethane, thermoplastic, fluorinated ethylene propylene (FEP), absorbable or resorbable polymers such as polyglycolic acid (PGA), polylactic acid (PLA), polycaprolactone (PCL), polyethyl acrylate (PEA), polydioxanone (PDS), and pseudo-polyamino tyrosine-based acids, extruded collagen, silicone, zinc, echogenic, radioactive, radiopaque materials, a biomaterial (e.g., cadaver tissue, collagen, allograft, autograft, xenograft, bone cement, morselized bone, osteogenic powder, beads of bone) any of the other materials listed herein or combinations thereof. Examples of radiopaque materials are barium sulfate, zinc oxide, titanium, stainless steel, nickel-titanium alloys, tantalum and gold.

Any or all elements of the ultra thin expanding stent device 2 and/or other devices or apparatuses described herein, can be, have, and/or be completely or partially coated with agents and/or a matrix a matrix for cell ingrowth or used with a fabric, for example a covering (not shown) that acts as a matrix for cell ingrowth. The matrix and/or fabric can be, for example, polyester (e.g., DACRON® from E. I. Du Pont de Nemours and Company, Wilmington, Del.), polypropylene, PTFE, ePTFE, nylon, extruded collagen, silicone or combinations thereof.

The elements of the ultra thin expanding stent device and/or other devices 2 or apparatuses described herein and/or the fabric can be used with cements or fillers, or filled and/or coated with an agent delivery matrix known to one having ordinary skill in the art and/or a therapeutic and/or diagnostic agent.

Examples of such cement and/or fillers includes bone chips, calcium sulfate, coralline hydroxyapatite, Biocoral, tricalcium phosphate, calcium phosphate, PMMA, bone morphogenic proteins, other materials described herein, or combinations thereof.

The agents within these matrices can include radioactive materials; radiopaque materials; cytogenic agents; cytotoxic agents; cytostatic agents; thrombogenic agents, for example polyurethane, cellulose acetate polymer mixed with bismuth trioxide, and ethylene vinyl alcohol; lubricious, hydrophilic materials; phosphor cholene; anti-inflammatory agents, for example non-steroidal anti-inflammatories (NSAIDs) such as cyclooxygenase-1 (COX-1) inhibitors (e.g., acetylsalicylic acid, for example ASPIRIN® from Bayer AG, Leverkusen, Germany; ibuprofen, for example ADVIL® from Wyeth, Collegeville, Pa.; indomethacin; mefenamic acid), COX-2 inhibitors (e.g., VIOXX® from Merck & Co., Inc., Whitehouse Station, N.J.; CELEBREX® from Pharmacia Corp., Peapack, N.J.; COX-1 inhibitors); immunosuppressive agents, for example Sirolimus (RAPAMUNE®, from Wyeth, Collegeville, Pa.), or matrix metalloproteinase (MMP) inhibitors (e.g., tetracycline and tetracycline derivatives) that act early within the pathways of an inflammatory response. Examples of other agents are provided in Walton et al, Inhibition of Prostoglandin E₂ Synthesis in Abdominal Aortic Aneurysms, Circulation, Jul. 6, 1999, 48-54; Tambiah et al, Provocation of Experimental Aortic Inflammation Mediators and Chlamydia Pneumoniae, Brit. J. Surgery 88 (7), 935-940; Franklin et al, Uptake of Tetracycline by Aortic Aneurysm Wall and Its Effect on Inflammation and Proteolysis, Brit. J. Surgery 86 (6), 771-775; Xu et al, Sp1 Increases Expression of Cyclooxygenase-2 in Hypoxic Vascular Endothelium, J. Biological Chemistry 275 (32) 24583-24589; and Pyo et al, Targeted Gene Disruption of Matrix Metalloproteinase-9 (Gelatinase B) Suppresses Development of Experimental Abdominal Aortic Aneurysms, J. Clinical Investigation 105 (11), 1641-1649 which are all incorporated by reference in their entireties.

The ultra thin expanding stent devices 2 can be laser cut, and/or non-laser cut. The ultra thin expanding stent device 2 can be laser cut in a partially opened pattern, then the ultra thin expanding stent device 2 can be loaded (e.g., crimped) onto a deployment tool 30. The loaded ultra thin expanding stent device 2 can have a smaller profile while plastically deforming the struts 14 past their limits.

The ultra thin expanding stent device 2 can be longitudinally segmented. Multiple ultra thin expanding stent devices 2 can be attached first end 6 to second end 8, and/or a single ultra thin expanding stent device 2 can be severed longitudinally into multiple ultra thin expanding stent devices 2.

Method of Use

The ultra thin expanding stent device 2 can be implanted in a bone, such as a compression fracture in a vertebra 42. The ultra thin expanding stent device 2 can be implanted in soft tissue, such as a herniated intervertebral disc 44.

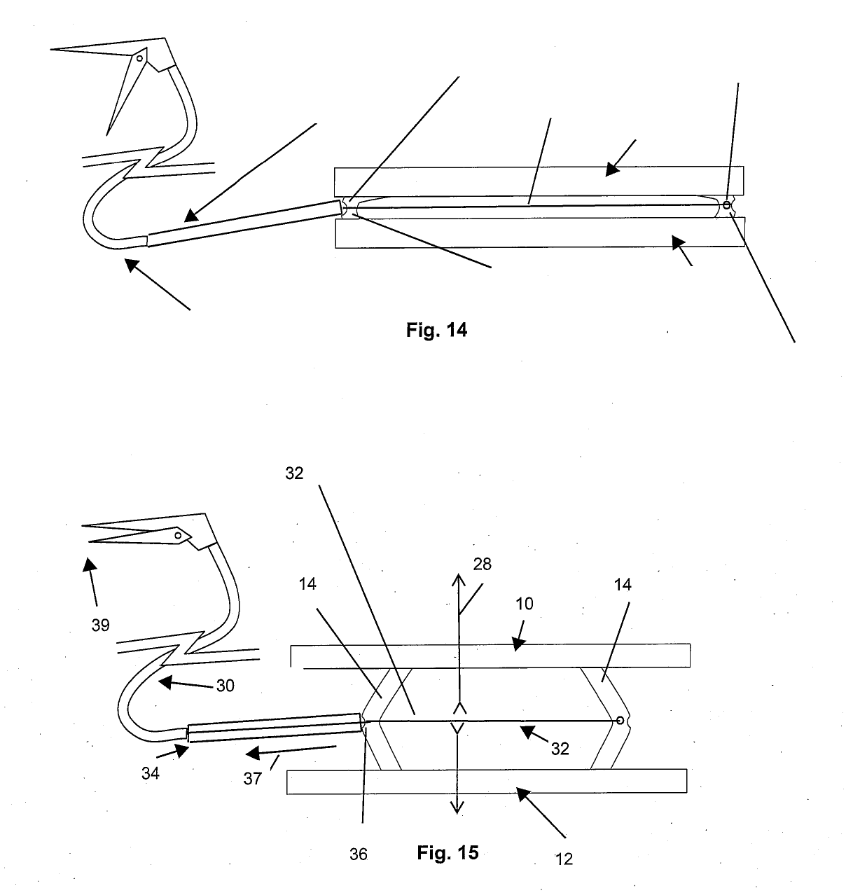

FIG. 14 illustrates that the ultra thin expanding stent device 2 can be loaded in a collapsed (i.e., unexpanded) configuration onto a deployment tool 30. The deployment tool 30 can have a handle 52 with a lever attached to a cable 32. The cable 32 can pass through a cable casing 34 to the ultra thin expanding stent device 2. The cable casing 34 can engage an engagement divot 22 in a proximal strut 14 in order to maintain its orientation during deployment, for example the first engagement divot 36 as illustrated in FIG. 14. The distal end of the cable 32 can be removably attached to the distal strut 14 by means of a distal engagement divot, for example the second engagement divot 38 as illustrated in FIG. 14.

FIG. 15 illustrates that the lever of the deployment tool 30 can be squeezed, as shown by the arrow 39, causing withdrawal, as shown by the arrow, of the cable 32 through the cable casing 34 and through the proximal or first engagement divot 36, thereby causing the ultra thin expanding stent device 2 to expand in height 37 as illustrated by the arrows 28.

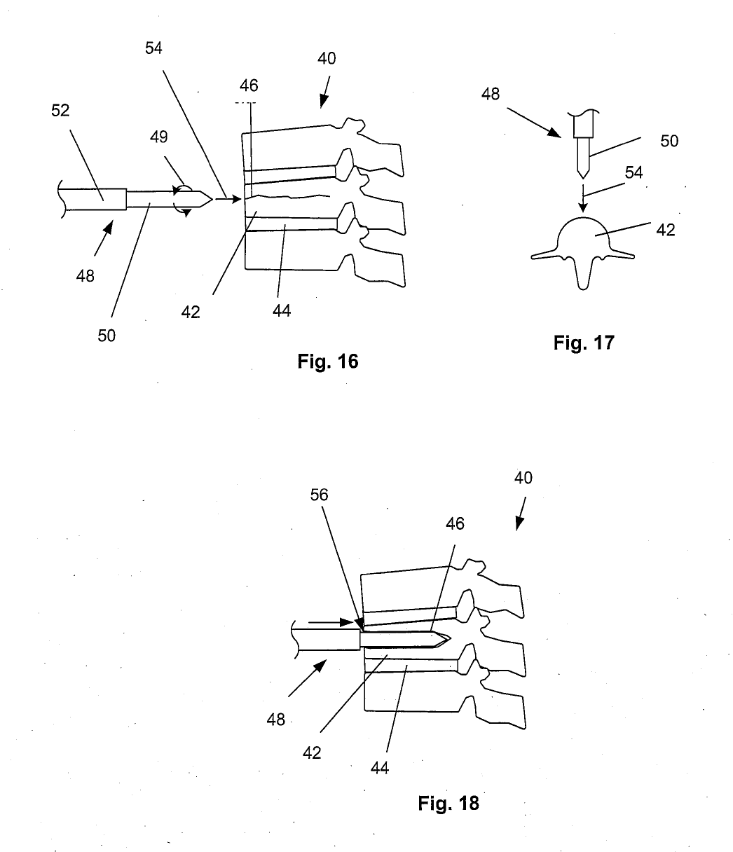

FIGS. 16 (side view) and 17 (top view) illustrate a vertebral column 40 that can have one or more vertebra 42 separated from the other vertebra 42 by discs 44. The vertebra 42 can have a damage site 46, for example a compression fracture.

An access tool 48 can be used to gain access to the damage site 46 and or increase the size of the damage site 46 to allow deployment of the ultra thin expanding stent device 2. The access tool 48 can be a rotating, as shown by the arrow 49, or vibrating drill 50 that can have a handle 52. The drill 50 can be operating, as shown by the arrows in FIGS. 16-18. The drill 50 can then be translated, as shown by arrow 54, toward and into the vertebra 42 so as to pass into the damage site 46.

FIG. 18 illustrates that the access tool 42 can be translated, as shown by arrow 54, to remove tissue at the damage site 46. The access tool 48 can create an access port 56 at the surface of the vertebra 42. The access port 56 can open to the damage site 46. The access tool 48 can then be removed from the vertebra 42.

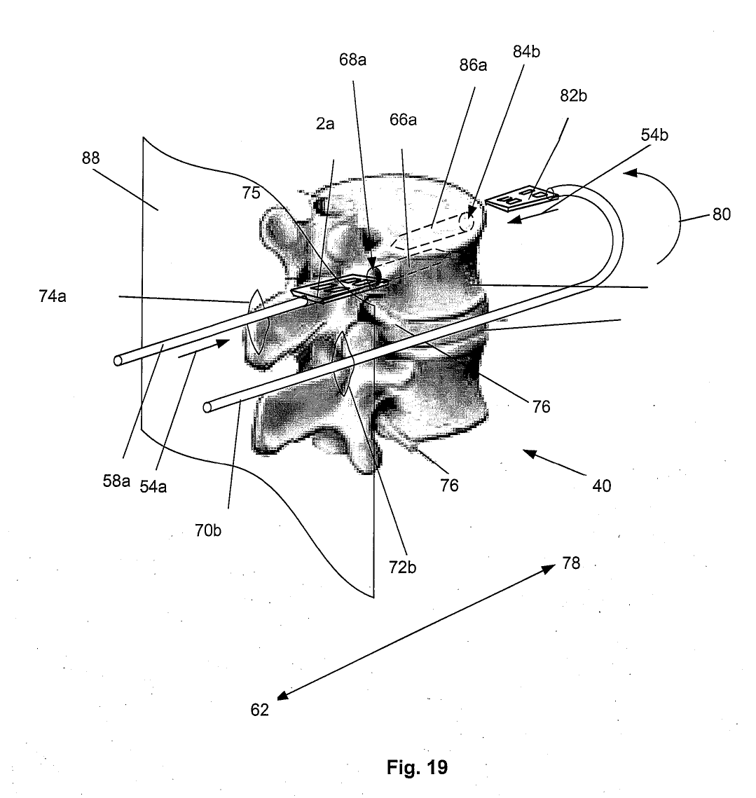

FIG. 19 illustrates that a first deployment tool 58a can enter through the subject's back. The first deployment tool 58a can enter through a first incision 60a in the skin 88 on the posterior 62 side of the subject near the vertebral column 40. The first deployment tool 58a can be translated, as shown by arrow 54, to position a first ultra thin expanding stent device 64a into a first damage site 66a. The first access port 68a can be on the posterior 62 side of the vertebra 42.

A second deployment tool 70b can enter through a second incision 72b (as shown) in the skin 88 on the posterior 62 or the first incision 74a. The second deployment tool 70b can be translated through muscle (not shown), around the spinal cord 87 and nerves 76, and anterior 78 of the vertebral column 40. The second deployment tool 70b can be steerable 80. The second deployment tool 70b can be steered, as shown by arrow 80, to align the distal tip of the second ultra thin expanding stent device 82b with a second access port 84b on a second damage site 86b. The second access port 84b can face anteriorly. The second deployment tool 70b can translate, as shown by arrow 54, to position the second ultra thin expanding stent device 2 in the second damage site 86b. The construction of the ultra thin expanding stent may allow for less invasive implantation as it may be deployed within the body from a single access point.

The device can be deployed using a guide pin. The device can be threaded, or otherwise advanced, over the guide pin to a target site.

An introducer tube or a guide member with a rectangular guide channel can be used to deliver the ultra thin expanding stent device 2 to a target site.

The vertebra 42 can have multiple damage sites 46 and ultra thin expanding stent devices 2 deployed therein. The ultra thin expanding stent devices 2 can be deployed from the anterior 78, posterior 62, both lateral, superior, inferior, any angle, or combinations of the directions thereof.

It should be noted that the ultra thin expanding stent devices 2 of the present invention may be deployed such that they expand in the width direction an do not change in height (e.g., by rotating the device 90 degrees.)

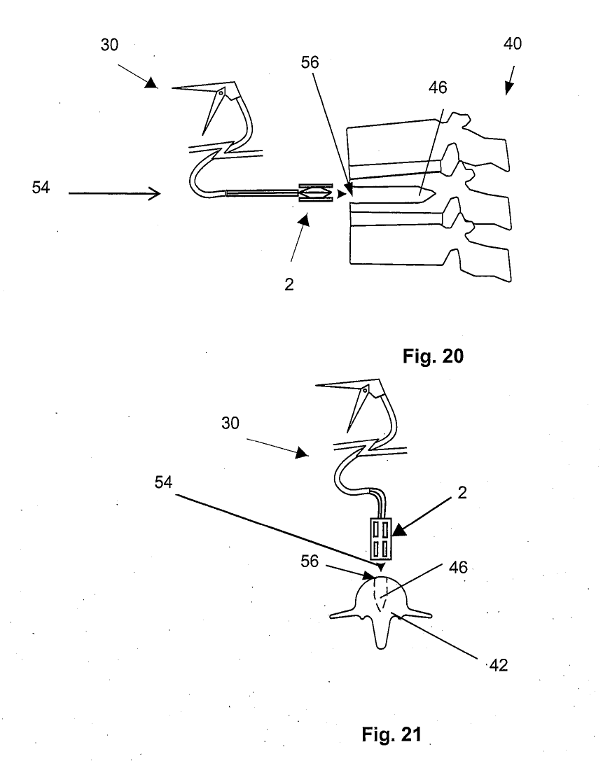

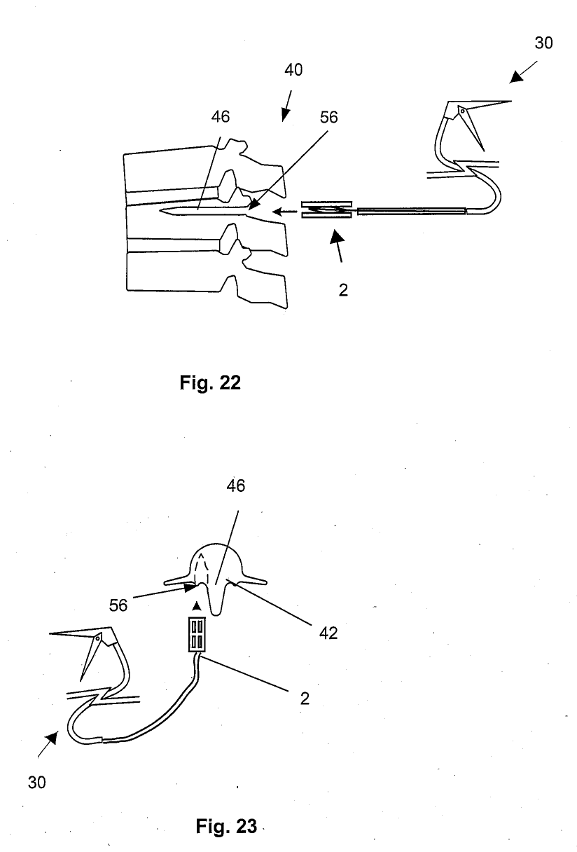

FIGS. 20 and 21 illustrate translating, as shown by arrow heads 54, the deployment tool 30 loaded with the ultra thin expanding stent device 2 through the access port 56 from the anterior 78 side of the vertebral column 40. FIGS. 22 and 23 illustrate that the deployment tool 30 can be deployed from the posterior 62 side of the vertebral column 40. The deployment tool 30 can be deployed off-center, for example, when approaching the posterior 62 side of the vertebral column 40, for example as illustrated in FIG. 23.

The ultra thin expanding stent 2 can be deployed within the vertebral body. The stent can be deployed between vertebra 42 (e.g., in a vertebral disc). The stent can be deployed over an end plate of the vertebral body.

The access port 56 can have an access port diameter 104. The access port diameter 104 can be from about 1.5 mm (0.060 in.) to about 40 mm (2 in.), for example about 8 mm (0.3 in.). The access port diameter 104 can be a result of the size of the access tool 48. After the ultra thin expanding stent device 2 is deployed, the damage site 46 can have a deployed diameter 106. The deployed diameter 106 can be from about 1.5 mm (0.060 in.) to about 120 mm (4.7 in.), for example about 20 mm (0.8 in.). The deployed diameter 106 can be greater than, equal to, or less than the access port diameter 104.

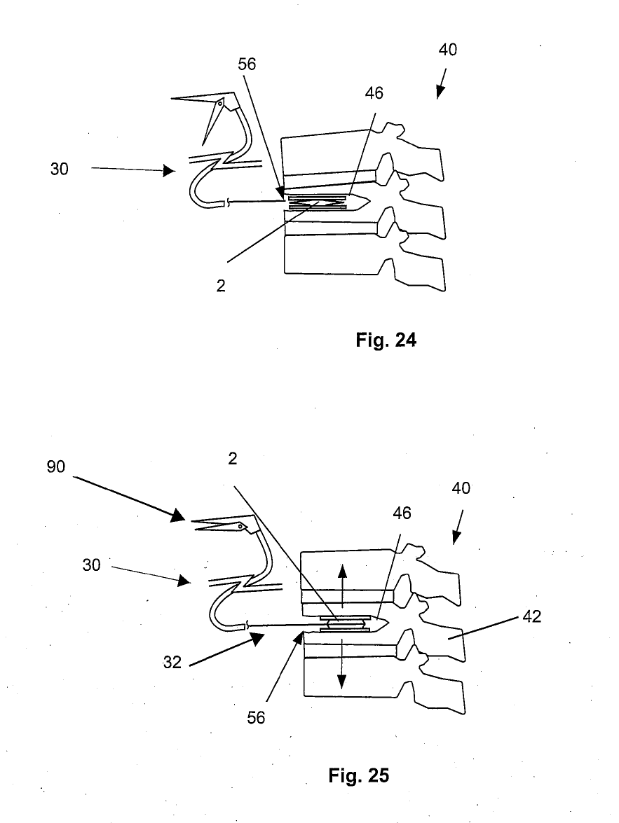

FIG. 24 illustrates that deployment tool 30 can position the ultra thin expanding stent device 2 in the vertebra and into the damage site 42.

FIG. 25 illustrates that the lever 90 on the deployment tool 30 can be withdrawn, thereby withdrawing the cable 32. The ultra thin expanding stent device 2 can expand, for example, due to the withdrawal of the cable 32. The cable 32 can be withdrawn until the ultra thin expanding stent device 2 is substantially fixed to the vertebra 42. The ultra thin expanding stent 2 device can reshape the vertebral column 40 to a more natural configuration during the expansion of the ultra thin expanding stent device 2.

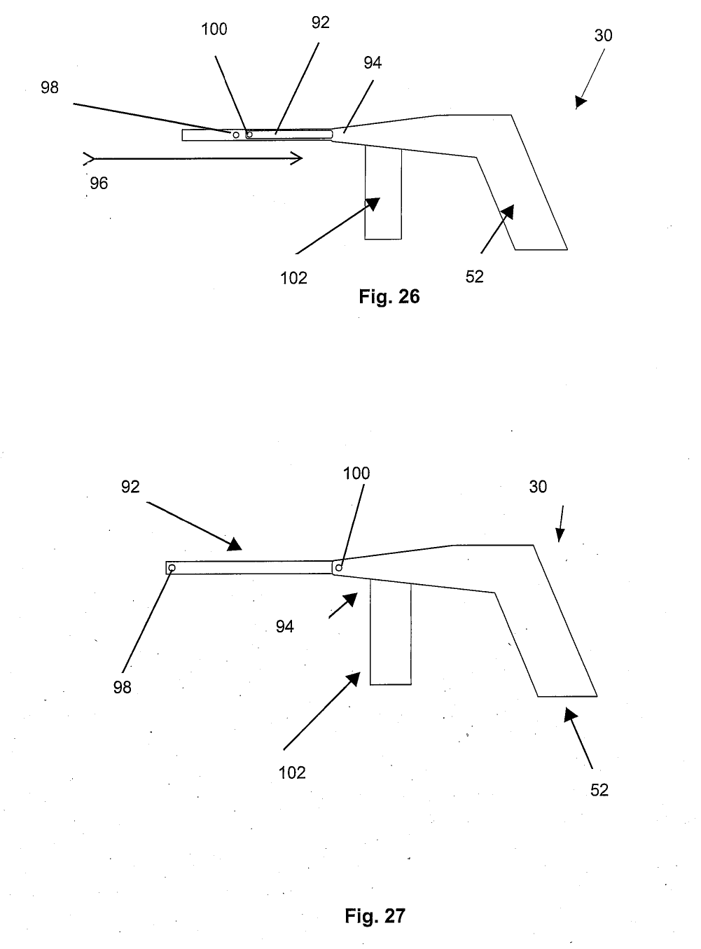

FIG. 26 illustrates that the deployment tool 30 can have a shaft 92 and a sleeve 94. The shaft can be slidably received, as shown by arrow, by the sleeve 96. The sleeve 94 can have a first pin 98. The shaft 92 can have a second pin 100. FIG. 26 illustrates that the shaft 92 can be connected to an actuator 102, which when withdrawn, can move the second pin 100 away from the first pin 98. This type of deployment tool 30 can be used to deploy ultra thin expanding stent devices 2 with struts that unfold outward 15, such as illustrated in FIGS. 5, 6, and 13.

FIG. 27 illustrates that the deployment tool 30 can also be configured to move the first 98 and second 100 pin closer together, when the actuator 102 is withdrawn. A deployment tool 30 can be used in which the first pin 98 can be attached to the shaft 92, and the second pin 100 can be attached to the sleeve 94 (e.g., a reverse arrangement from the deployment tool 30 illustrated in FIG. 26). The deployment tool 30 can be used, for example, to deploy the ultra thin expanding stents 2 with struts 14 that unfold inward toward the center of the ultra thin expanding stent device, such as the type illustrated in FIGS. 11 and 12.

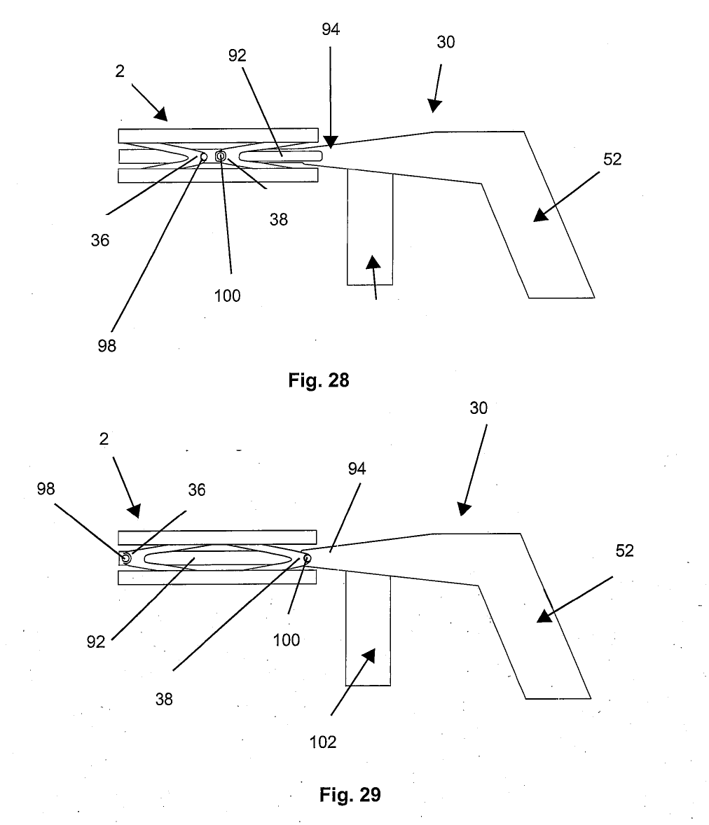

FIGS. 28 and 29 illustrate that the ultra thin expanding stent device 2, for example in an unexpanded configuration, can be loaded on the shaft 92 of a deployment tool 30. The first end of the ultra thin expanding stent device 2 can be received by and/or interference fit in the first pin 98, for example by engagement in a first engagement divot 36 in a strut 14. The second end 8 of the ultra thin expanding stent device 2 can be received by and/or interference fit in the second pin 100, for example by connection to a second engagement divot 38 in a strut 14.

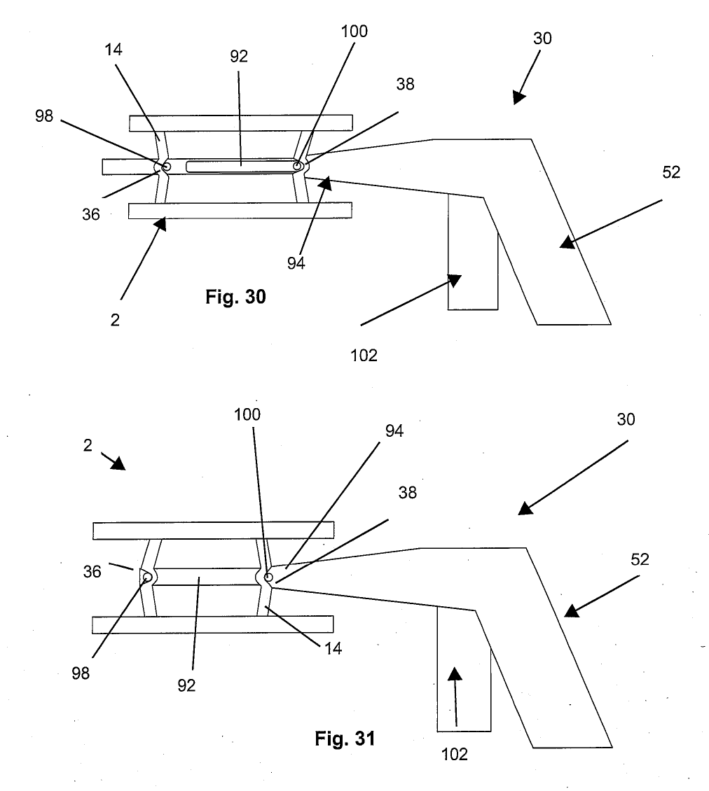

FIGS. 30 and 31 illustrate that the ultra thin expanding stent device 2 can be deployed by use of a deployment tool 30, for example to press the folded apexes of the struts 14 apart, such as illustrated by FIG. 30, or to pull the folded apexes of struts 14 together, as illustrated by FIG. 31. The ultra thin expanding stent device 2 can thereby be resiliently and/or deformably forced into an expanded configuration. The ultra thin expanding stent device 2 can then be released from the deployment tool 30, for example, by sliding the shaft 92 away from the sleeve 94.

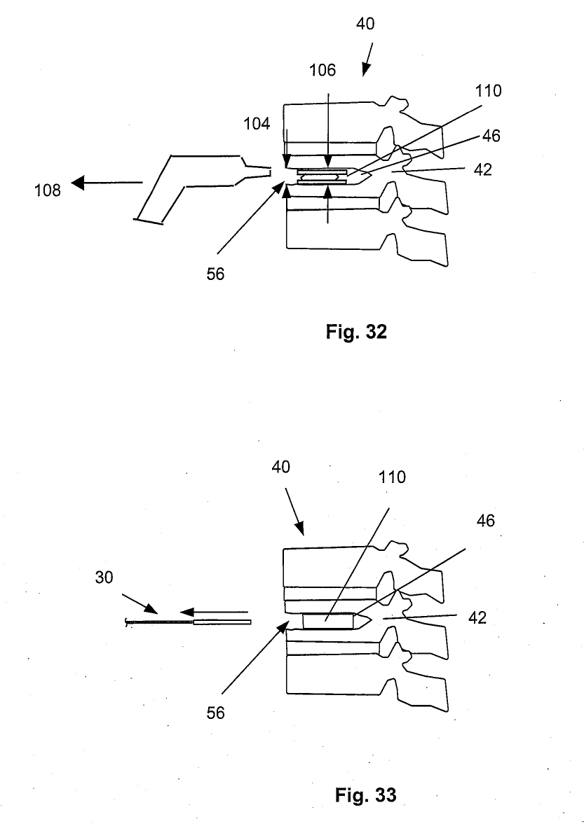

FIGS. 32 and 33 illustrate that the insertion tool or tools 108 or deployment tool 30 can be removed after being used to expand the ultra thin expanding stent device, leaving the ultra thin expanding stent device 2 substantially fixed to the vertebra 42 at the damage site 46. During the ensuing healing process, the bone can grow into the plate openings 20, further securing the ultra thin expanding stent device 2 at the repair site.

U.S. Provisional Application Ser. Nos. 60/612,001, filed 21 Sep. 2004; 60/611,972, filed 21 Sep. 2004; 60/612,723, filed 24 Sep. 2004; 60/612,724, filed 24 Sep. 2004; and 60/612,728, filed 24 Sep. 2004 are herein incorporated by reference in their entireties.

It is apparent to one skilled in the art that various changes and modifications can be made to this disclosure, and equivalents employed, without departing from the spirit and scope of the invention. Elements shown with any embodiment are exemplary for the specific embodiment and can be used on other embodiments within this disclosure.

We claim:

1. An expandable support device for spinal repair, comprising:
a substantially flat first plate;
a substantially flat second plate; and
a first strut attaching the first plate and second plate, wherein the first strut has contracted and expanded configurations, and wherein when the first strut is in the contracted configuration, the first plate is closer to the second plate than when the first strut is in the expanded configuration.

2. The device of claim 1, further comprising a second strut attaching the first plate and second plate, wherein the second strut has contracted and expanded configurations, and wherein when the second strut is in the contracted configuration, the first plate is closer to the second plate than when the second strut is in the expanded configuration.

3. The device of claim 1, wherein the first strut is configured to constrain the first plate to uni-dimensional translational motion with respect to the second plate.

4. The device of claim 1, wherein the first plate is at the top of the expandable support device.

5. The device of claim 4, wherein the second plate is at the bottom of the expandable support device.

6. The device of claim 1, wherein the first plate has two parallel plate faces.

7. The device of claim 6, wherein the second plate has two parallel plate faces.

8. The device of claim 1, wherein the first strut is configured to fold.

9. The device of claim 8, wherein the first and second plates define a longitudinal axis therebetween, and wherein the first strut is oriented at an intersection angle with respect to the longitudinal axis, wherein the intersection angle is about 90 degrees when the first strut is in the expanded configuration.

10. The device of claim 1, wherein the first strut has a thinning.

11. The device of claim 10, wherein the thinning is about halfway along the length of the first strut.

12. The device of claim 1, wherein the first plate has a plate opening therethrough.

13. A method for deploying an expandable support device in a spine, the expandable support device having a longitudinal axis, a first end and a second end, comprising:
deploying the device into a target site; expanding the device, wherein expanding comprises applying forces to the device such that the first end and the second end angularly rotate with respect to the longitudinal axis, and wherein the first end is substantially parallel with the second end before the expanding, and wherein the first end is substantially parallel with the second end after the expanding.

14. The method of claim 13, wherein the target site is a vertebra.

15. The method of claim 13, wherein the target site is a vertebral end-plate.

16. The method of claim 13, wherein the target site is a vertebral disc.

17. The method of claim 13, wherein expanding the device comprises increasing a height of the device while a width of the device remains substantially constant.

18. The method of claim 13, where expanding the device comprises increasing a width of the device while a height of the device remains unchanged.

19. The method of claim 13, where deploying the device comprises inserting a guide pin to the target site, and advancing the device over the guide pin.

20. The method of claim 13, wherein deploying the device comprises deploying the device through a tube.

21. The method of claim 20, wherein the tube comprises a lumen having a rectangular cross-sectional shape.
